(19) United States
(12) Patent Application Publication
(10) Pub. No.: US 2005/0143345 A1
(43) Pub. Date: Jun. 30, 2005

(54) Cosmetic scar management composition

(75) Inventor: Craig Julian Hardy, Cheshire (GB)

Correspondence Address: HOFFMANN & BARON, LLP, 6900 JERICHO TURNPIKE, SYOSSET, NY, 11791, US

(73) Assignee: MEDTRADE PRODUCTS LIMITED, Crewe (GB)

(21) Appl. No.: 10/498,637

(22) PCT Filed: Dec. 13, 2002
(86) PCT No.: PCT/GB02/05686

(30) Foreign Application Priority Data
Dec. 14, 2001 (GB) 0129886.8

(51) Int. Cl. A61K 31/695; A61K 7/06; A61K 7/11

(52) U.S. Cl. 514/63; 424/70.12

(57) ABSTRACT

A scar management composition for reducing the cosmetic severity of scars which comprises at least one silicone component, water and at least one thickening agent.

The present invention relates to a composition which finds particular utility in scar management.

It has been known for some years that the cosmetic severity of tissue scarring as a result of medical treatment and/or injury can be greatly reduced by the application of a silicone-containing sheet to the skin's surface. The mode of action remains unclear.

The first silicone-containing sheets used in order to reduce the visual severity of tissue scarring, based on a rubber containing formulation, were developed approximately 25 years ago and met with limited success since they were difficult to use. These silicone rubber sheets were secured on the skin surface with adhesive tape thereby forming a sealed enclosure. Typically, the silicone rubber sheet would be secured in place for 30 days during which time the skin would ‘remodel’ itself to a large degree giving rise to a decrease in the cosmetic severity of the scar tissue.

Several theories have been postulated regarding the mode of action of the silicone layers. These include the moisture barrier properties of silicone, the leaching of low molecular weight silicone into the skin and the effect of the ionic content of the silicone.

Despite these silicone rubber sheets being expensive, they were widely used even though the location of their use on the skin surface was limited by their lack of flexibility.

The aforesaid silicone rubber sheets have since been replaced with softer self-adhesive silicone gel sheets, which possess a greater degree of flexibility by virtue of their polymeric oil-containing network structure. Therefore, although easier to apply, their use remains limited as a result of their sheet-like nature

More recently liquid silicone compositions have been introduced. These are supplied in a tube and are specifically designed to be applied to awkward areas of the skins surface. Example of said compositions include KELO-COTE(™) or with a roller ball applicator, CICACARE(™). Unlike the previously described sheets, which are intended to be worn for a period of approximately 30 days, these liquid silicone compositions require re-application every few hours as the silicone layer located over the wound is thinner. However, these compositions remain sticky and tack-y after application, some fail to dry, whilst others take 10 to 30 minutes to dry.

It is desirable therefore to provide a composition which delivers the aforesaid cosmetic scar severity reduction and which is more convenient to use.

According to a first aspect of the present invention there is provided a scar management composition comprising at least one silicone component, water and at least one thickening agent.

Advantageously, the composition of the present invention is in the form of a surprisingly stable gel Which is easy to manufacture and as such is suitable for application to all skin areas, but it is particularly useful for the application to areas to which it is difficult to apply sheet material.

By gel it is meant a viscous, at least partially rigid material which can be manipulated to form a thin film.

A further advantage of the composition of the present invention lies with its ability to deliver moisture to the wound immediately on application by virtue of the composition's water content, thus) eliminating the need, as is the case with prior art silicone-containing sheets, for moisture to develop under the sheet thus giving an immediate skin softening and moisturising effect. The water content of the compositions of the present invention ultimately results in a more efficient and effective treatment.

A still further advantage of the composition of the present invention is it's rapid drying time, typically of about 2 minutes, thereby eliminating the undesirable tack of previously used liquid compositions which have a much longer drying time, typically in the order of 10 to 30 minutes.

Surprisingly, the composition of the present invention results in a greater reduction in the visual severity of a scar than that seen with previous methods of scar management.

Accordingly, the composition of the present invention is not only much easier and quicker to use than its predecessors but results in a greater reduction in the visual severity of scar tissue.

The silicone component preferably constitutes from 0.01% to 80%, more preferably from 1% to 50% and most preferably from 5% to 30% by weight of the composition of the present invention.

Suitable silicone components for use in the composition of the present invention include any of the following eiher alone or in combination:—silicone, silicone oil, silicone fluids, silicone solvents, silicone gels, silicone grease, silicone elastomer, or cross polymer, silicone gum, silicone adhesive silicone emulsifier, silicone wax emulsifier and silicone copolymer.

Preferably, the silicone component of the present invention is a silicone elastomer and/or a silicone gum. Advantageously this gives rise to a composition having a longer wear time than currently available scar management products. Table 3, shows how the inclusion of silicone elastomer increases the wear time of the product.

The thickening agent(s) preferably constitutes from 0.01% to 50%, more preferably from 0.01% to 10%, still more preferably from 0.01% to 0.99% and most preferably fro, 0.01% to 0.75% by weight of the composition of the present invention.

Suitable thickening agents include any of the foilowing either alone or in combination:—polyaciylic acid thickeners such as Carbopol, polyacrylic acid derivatives, polsrvyl alcohol (PVA, PVOH), pobvyl pyrrolidine (PVP), PVP/PVA copolymer, hydroxyethyl cellulose, acrylatepolymer, alkyl acrylates cross copolymer, hydrolysed protein, cetearyl alcohol, plant polysaccharide, microbial polysaccharide, animal polysaccharide, carboxymethyl cellulose, methyl cellulose, alginate, chitin/chitosan, hyaluronic acid, collagen, pectin, gelatine, starch and starch copolymer.

Preferably water constitutes from 2% to 99.8%, by weight of the composition of the present invention.

Preferably the composition of the present, invention shall contain at least one volatile solvent and most preferably at least one volatile organic solvent, for example isopropyl alcohol (IPA), ethanol, methanol, acetone, low molecular weight silicone, cyclomethicone, volatile silicone or silicone solvent, ethoxydiglycol, hexane.

The said solvent may constitute from 0% to 80%, preferably from 5% to 80% and most preferably from 10.1% to 80% by weight, of the composition of the present invention.

Preferably the composition of the present invention shall comprise at least one neutralising agent which can be mineral and/or organic in nature. Any suitable base may be used, for example sodium hydroxide, potassium hydroxide, triethanolamine and any suitable inorganic base.

In addition to the foregoing the composition of the present invention may also comprise any of the following, either alone or in combination:—active agents such as antibacterial agents, surfactants, antimicrobial agents, essential oil skin occlusion agents, copolymer and skin moisturisers, skin healing agents, scar healing agents, growth factors, or any compound which effects the level of growth factors in the skin, wound or scar, herbal agents, plant oils, essential oils or other plant extracts.

According to a second aspect of the present invention therefore there is provided a method of preparing a scar management composition comprising mug together at least two formulations wherein the first formulation comprises water and at least one thickening agent and the second formulation comprises at least one silicone component.

The first formulation may be a viscous water-based hydrogel which is formed when water containing at least one thickening agent typically in total 1%, by weight, is vigorously stirred The resulting viscous hydrogel is subsequently neutralised using sodium hydroxide, for example. The first formulation may optionally contain at lest one silicone component. The fist formulation may also contain active agents such as antibacterial agents, antimicrobial agents and/or skin moisturising agents.

The second formulation may be a mixture of two independent parts.

The first part of the second formulation may comprise volatile organic solvent(s) and any organic solvent soluble components such as one or more skin moisturisers.

The second part of the second formulation may comprise any of the aforesaid silicone components along w any surfactant and copolymer.

During preparation of the composition of the present invention the first formulation may be added to the second formulation with vigorous mixing, again until a homogeneous hydrogel ensues.

In order that the present invention be more readily understood the composition of the present invention now will now be described further by way of example only with relation to the following examples:

### EXAMPLE 1

Silmogen Masterbatch ethanol and nusil silicone grease were pre-mixed to form the first formulation as a homogeneous mixture.

Carbopol ultrex was added to water with continuous sting, furnishing the first formulation as a homogeneous hydrogel which was neutralised with 2% aqueous soluton of sodium hydroxide.

The first fomulation was then added to the second formulation with vigorous stirring to furnish a homogenous gel the composition of the present invention.

| Formulation | Component | Function | Quantity |
|---|---|---|---|
| First | Water | Solvent | 200 g |
| | Carbopol Ultrex | Thickening Agent | 2 g |
| | NaOH (2% | Neutralising Agent | 2 wt % |
| | solution) | | of total comp. A |
| Second | Silmogen | Silicone | 54 g |
| | Masterbatch | Component | |
| | Ethanol | Organic Solvent | 18 g |
| | Nusil | Silicone Grease | 6 g |

NB: Drying time (average of 3 tests) 93 seconds.

### EXAMPLE 2

The composition was prepared in the aforesaid manner

| Formulation | Component | Function | Quantity |
|---|---|---|---|
| First | Water | Solvent | 200 g |
| | Carbopol 914 | Thickening Agent | 1.5 g |
| | Neutrol TE | Neutralising Agent | to pH 7 |
| Second | Ethanol | Organic Solvent | 100 g |
| | Silmogen Carrier | Silicone Solvent | 100 g |
| | Dimethicone Blend | Silicone | 20-25 g |
| | | Component | |

NB: Drying time 23 seconds.

### EXAMPLE 3

The composition was prepared in the aforesaid manner

| Formulation | Component | Function | Quantity |
|---|---|---|---|
| First | Water | Solvent | 190 g |
| | Medical Grade | Moisturising | 10 g |
| | Aloe Vera Gel | Agent | |
| | Carbopol 914 | Thickening Agent | 5 g |
| | Neutrol TE | Neutralising Agent | to pH 7 |
| Second | Ethanol | Organic Solvent | 200 g |
| | Silmogen Carrier | Silicone Solvent | 125 g |
| | Dimethicone Blend | Silicone | 20-75 g |
| | | Component | |

NB: Drying time 98 seconds.

### EXAMPLE 4

The composition was prepared in the aforesaid manner

| Formulation | Component | Function | Quantity |
|---|---|---|---|
| First | Water | Solvent | 200 g |
| | Carbol 914 | Thickening Agent | 5 g |
| | Salicylic Acid | Antibacterial | 0.5 g |
| | | Agent | |
| | Neutrol TE | Neutralising Agent | to pH 7 |
| Second | Ethanol | Organic Solvent | 200 g |
| | Silmogen Carrier | Silicone Solvent | 125 g |
| | Dimethicone Blend | Silicone | 20-75 g |
| | | Component | |

NB: Drying time - not available.

### EXAMPLE 5

The composition was prepared in the aforesaid manner

| Formulation | Component | Function | Quantity (g) |
|---|---|---|---|
| First | Water | Solvent | 247.55 |
| | Ethanol | Organic Solvent | 108.35 |
| | Carbopol 980NF | Thickener | 3.20 |
| | Nipagin M | Preservative | 0.29 |
| | Nipasol M | Preservative | 0.07 |
| Second | Elastomer 10 | Silicone | 33.85 |
| | | component | |
| | Cyclomethicone 5 | Silicone | 22.44 |
| | | component | |
| | Isopropyl | Solubilising agent | 4.25 |
| | Myristate | | |

The composition was neutralised to between pH 7.3 and 7.6 using sodium hydroxide.
N.B. Drying time 80 seconds.

### EXAMPLE 6

The composition was prepared in the aforesaid manner

| Formulation | Component | Function | Quantity (g) |
|---|---|---|---|
| First | Water | Solvent | 247.55 |
| | Ethanol | Organic Solvent | 108.35 |
| | Carbopol 980NF | Thickening Agent | 4.6 |
| | Nipagin M | Preservative | 0.29 |
| | Nipasol M | Preservative | 0.07 |
| Second | Dow Corning | Silicone | 56.5 |
| | silicone fluid | component | |

Drying time - not available.

### EXAMPLE 7

Unlike the previous examples the composition described below does not contain any organic solvents.

Carbopol 940 was added to water, with vigorous siring. The resulting hydrogel was neutralised using sodium hydroxide, thus furnishing the first formulation.

The first formulation was then stirred vigorously while dimethicone blend 20, the second formulation, was introduced.

The resulting gel was mixed for a three minutes providing the composition of the present invention.

| Formulation | Component | Function | Quantity |
|---|---|---|---|
| First | Water | Solvent | 200 g |
| | Carbopol 940 | Thickening Agent | 1.5 g |
| | NaOH | Neutralising Agent | to pH 7 |
| Second | Dimethicone blend | Silicone | 20 g |
| | 20 | Component | |

NB: Drying time 435 seconds.

### EXAMPLE 8

In addition to the components of examples 1 to 4the composition described below contains emulsifiers.

Carbopol 980 was added to water. This mixture was then pre-heated to 50° C. and a mixture of stearyl alcohol and trimethylstearyloxysilane was introduced providing the first formulation as an emulsion, which was then allowed to cool.

Dimethicone blend 20 and ethanol were mixed together resulting in the second formulation.

The second fomulation was then introduced to a vigorously stirred first formulation.

| Formulation | Component | Function | Quantity |
|---|---|---|---|
| First | Water | Solvent | 168 g |
| | Carbopoly 980 | Thickening Agent | 2 g |
| | Silky wax 10 (a | Silicone component | 2 g |
| | mixture of stearyl | | |
| | alcohol and | | |
| | trimethylstearyloxy | | |
| | silane) | | |
| Second | Ethanol | Organic Solvent | 10% of |
| | | | component |
| | | | phase A |
| | Dimethicone blend 20 | Silicone Component | 10% of |
| | | | component |
| | | | phase A |

NB: Drying time 360 seconds.

Clinical cast studies have been performed to assess the effectiveness of the scar management compositions of the present invention.

In the following case study the patient was asked using the guidelines exemplified in Table 1, to comment as to the appearance and feel of each scar following treatment with compositions described herein.

TABLE 1

| Questions | Results |
|---|---|
| 1. Has the scar improved since the last assessment? | Yes or No |
| 2. Rate its appearance on a scale of 1 to 5 | 1 to 5 |
| (with 1 as the best and 5 | |
| as the worst)? | |
| 3. Rate its flexibility on a scale of 1 to 4 | 1 to 5 |
| (with 1 as the best and 5 | |
| as the worst)? | |
| 4. Rate its softness on a scale of 1 to 5 | 1 to 5 |
| (with 1 as the best and 5 as | |
| the worst)? | |
| 5. Does it feel irritated? Rate this | 1 to 5 |
| on a scale of 1 to 5 (with 1 as the | |
| best and 5 as the worst)? | |
| 6. Is it better or worse than the scar it | Better or Worse |
| was originally paired against? | |

Case Study

A patient had four fatty cysts surgically removed from under the surface layer of the skin on the arm Following surgery the wounds were-sutured closed and dressings with Jelonet™ and a gauze bandage were applied thereto, The patient was asked to minimise the movement of the arm and keep it elevated for 24 hours after the operation.

The sutures were removed 10 days after surgery.

Three days after the removal of the sutures the scars appeared dry and red.

The patient was asked to apply a scar management composition according to the present invention to two of the scars (scar 1 and scar 3) each morning and after every bath or shower.

The other two scars (scar 2 and scar 4) were not treated.

The scars were assessed 12 days, 26 days and 68 days from the first treatment.

The results of the clinical case study are shown in Table 2.

TABLE 2

| Ques- | After 12 days | | | | After 26 days | | | | After 68 days | | | |
|---|---|---|---|---|---|---|---|---|---|---|---|---|
| tion | scar 1 | scar 2 | scar 3 | scar 4 | scar 1 | scar 2 | scar 3 | scar 4 | scar 1 | scar 2 | scar 3 | scar 4 |
| 1. | Yes | Yes | Yes | Yes | Yes | No, | Yes | No, it got | Yes | No | Yes | No |
| | | | | | | maybe | | worse, more | | | | |
| | | | | | | slightly | | red and more | | | | |
| | | | | | | softer | | raised | | | | |
| 2. | 3 | 3 | 4 | 5 | 2 | 3 | 3 | 5 | 1 | 3 | 2 | 5 |
| 3. | 2 | 4 | 3 | 3 | 1 | 3 | 2 | 2 | 1 | 2 | 1 | 3 |
| 4. | 2 | 4 | 3 | 3 | 1 | 3 | 2 | 2 | 1 | 2 | 1 | 3 |
| 5. | 3 | 3 | 2 | 4 | 1 | 2 | 1 | 5 | 1 | 2 | 1 | 4 |
| 6. | Very | Very | Same | Same | Better in | Worse in | Considerably | Considerably | Considerably | Worse in | Considerably | Considerably |
| | slightly | slightly | as | as | colour and | colour and | better than | worse than | better in | colour and | better than | worse than |
| | better | worse | scar 4 | scar 3 | softness/ | softness/ | scar 4 in | scar 3 in | colour and | softness/ | scar 4 in | scar 3 in |
| | than | than | | | flexibility | flexibility | colour | colour, | softness/ | flexibility | colour, | colour, |
| | scar 2 | scar 1 | | | than scar 2 | than scar 1 | | height and | flexibility | than scar 1 | height and | height and |
| | | | | | | | | irritation | than scar 2 | | irritation | irritation |

TABLE 3

| | | Elastomer | | | | | | | |
|---|---|---|---|---|---|---|---|---|---|
| | | or gum | Wear Time (hours) | | | | | | |
| Example | Format | present? | 1 | 2 | 4 | 6 | 8 | 10 | 12 |
| Competitor | — | Neither | Pass | Pass | Fail | | | | |
| Product | | | | | | | | | |
| Kelocote ™ | | | | | | | | | |
| (control) | | | | | | | | | |
| Example 6 | Hydrogel | Neither | Pass | Pass | Fail | | | | |
| Example 5 | Hydrogel | Elastomer | Pass | Pass | Pass | Pass | Pass | Pass | Fail |

Test Method Summary
0.07 g of test material is applied to a 5 cm² area of skin.

The area is wetted with water after 1 hour, 2 hours, 4 hours and then every 2 hours.

The product ‘passes’ if the water after forms an elevated bubble indicated that a significant amount of silicone is still present.

The product ‘fails’ if the water does not form a elevated bubble indicated that little if any silicone is still present.

It is of course to be understood that the invention is not intended to be restricted to the details of the above embodiments which are described by way of example only.

1. A scar management composition comprising at least one silicone component, water, and at least one thickening agent.

2. A scar management composition according to claim 1, wherein the silicone component constitutes from 0.01% to 80%, by weight, of the total composition.

3. A scar management composition according to claim 2, wherein the silicone constitutes from 1% to 50%, by weight, of the total composition.

4. A scar management composition according to claim 3, wherein the silicone constitutes from 5% to 30%, by weight, of the total composition.

5. A scar management composition according to claim 1, wherein the said silicone component comprises any of the following either alone or in combination: silicone, silicone oil, silicone fluid, silicone solvent, silicone gel, silicone grease, silicone elastomer or crosspolymer, silicone gum, silicone adhesive, silicone emulsifier, silicone wax emulsifier, silicone resin, organo-modified silicone polymer or other silicone copolymer.

6. A scar management composition according to claim 1, wherein the said silicone component comprises any of the following either alone or in combination: silicone elastomer or crosspolymer, silicone gum, silicone resin, organo-modified silicone polymer or silicone adhesive.

7. A scar management composition according to claim 1, wherein the said silicone component is a silicone gum.

8. A scar management composition according to claim 1, wherein the said silicone component is a silicone elastomer or crosspolymer.

9. A scar management composition according to claim 1, wherein the said silicone component is an organo-modified silicone polymer.

10. A scar management composition according to claim 1, wherein said at least one thickening agent constitutes from 0.01% to 50%, by weight, of the total composition.

11. A scar management composition according to claim 6, wherein said at least one thickening agent constitutes from 0.01% to 10%, by weight, of the total composition.

12. A scar management composition according to claim 11, wherein said at least one thickening agent constitutes from 0.01% to 0.99%, by weight, of the total composition.

13. A scar management composition according to claim 12, wherein said at least one thickening agent constitutes from 0.01% to 0.75%, by weight, of the total composition.

14. A scar management composition according to claim 1, wherein said at least one thickening agent comprises any of the following either alone or in combination: polyacrylic acid thickener, polyacrylic acid derivative, polyvinyl alcohol, polyvinyl pyrrolidine, polyvinyl pyrrolidine/polyvinyl acrylate copolymer, hydroxyethyl cellulose, acrylatepolymer, alkyl acrylate cross copolymer, hydrolysed protein, cetearyl alcohol, plant polysaccharide, microbial polysaccharide, animal polysaccharide, carboxymethyl cellulose, methyl cellulose, alginate, chitan/chitosan, hyaluronic acid, collagen, pectin, gelatine, starch or starch copolymer.

15. A scar management composition according to claim 1, wherein the water constitutes from 2% to 99.8%, by weight, of the total composition.

16. A scar management composition according to claim 1, wherein the composition further comprises at least one volatile solvent.

17. A scar management composition according to claim 1, wherein the composition further comprises at least one volatile organic solvent.

18. A scar management composition according to claim 16, wherein the said at least one solvent comprises any of the following either alone or in combination: isopropyl alcohol, ethanol, methanol, acetone, low molecular weight silicone, cyclomethicone, volatile silicone, or silicone solvent, ethoxydiglycol, or hexane.

19. A scar management composition according to claim 16, wherein said at least one volatile solvent constitutes from 0% to 80%, by weight, of the total composition.

20. A scar management composition according to claim 19, wherein said at least one solvent constitutes from 5% to 80%, by weight, of the total composition.

21. A scar management composition according to claim 20, wherein said at least one solvent constitutes from 10.1% to 80%, by weight, of the total composition.

22. A scar management composition according to claim 1, wherein the composition further comprises at least one neutralizing agent.

23. A scar management composition according to claim 22, wherein the said at least one neutralizing agent is a mineral and/or is organic in nature.

24. A scar management composition according to claim 22, wherein the said at least one neutralizing agent comprises any of the following either alone or in combination: sodium hydroxide, potassium hydroxide or triethanolamine.

25. A scar management composition according to claim 1, wherein the composition further comprises any of the following either alone or in combination: antibacterial agent, antimicrobial agent, antimicrobial essential oil, skin occlusion agents, skin moistuzizers, skin healing agents or scar healing agents, growth factor or any compound which affects the level of growth factors in the skin, wound or scar, herbal agents, plant oils, essential oils or other plant extract.

26. A scar management composition according to claim 1, wherein the composition further comprises skin occlusion agents.

27. A scar management composition according to claim 1, wherein the composition further comprises at least one skin moisturizer.

28. A scar management composition according to claim 1, wherein the composition further comprises any of the following either alone or in combination: skin healing agents, scar healing agents, growth factors or any compound which affects the level of growth factors in the skin, wound or scar.

29. A scar management composition according to claim 1, wherein the composition further comprises any of the following either alone or in combination: herbal agents, plant oils, essential oils or other plant extracts.

30. A method of.preparing a scar management composition comprising mixing together at least two formulations, wherein the first formulation comprises water and at least one thickening agent and a second formulation comprises at least one silicone component.

31. A method of preparing a scar management composition according to claim 30, wherein the said first formulation is a water-based hydrogel.

32. A method of preparing a scar management composition according to claim 31, wherein the water-based hydrogel is neutralized.

33. A method of preparing a scar management composition according to claim 30, wherein said first formulation, comprises any of the following either alone or in combination: at least one antibacterial or antimicrobial agent, and/or at least one skin moisturizing agent.

34. A method of preparing a scar management composition according to claim 30, wherein said second formulation is a mixture of two independent parts.

35. A method of preparing a scar management composition according to claim 30, wherein at least one of the said two formulations comprises at least one volatile solvent.

36. A method of preparing a scar management composition according to claim 30, wherein one part of the second formulation comprises said at least one silicone component.

37. A method of preparing a scar management composition according to claim 30, wherein the second formulation is a homogeneous gel.

38. (canceled)

39. A scar management composition according to claim 17, wherein the said at least one solvent comprises any of the following either alone or in combination: isopropyl alcohol, ethanol, methanol, acetone, low molecular weight silicone, cyclomethicone, volatile silicone or silicone solvent, ethoxydiglycol, or hexane.

40. A scar management composition according to claim 17, wherein said at least one volatile solvent constitutes from 0% to 80%, by weight, of the total composition.

41. A scar management composition according to claim 18, wherein said at least one volatile solvent constitutes from 0% to 80%, by weight, of the total composition.
